(19) United States
(12) Patent Application Publication
(10) Pub. No.: US 2026/0102149 A1
(43) Pub. Date: Apr. 16, 2026

(54) CLIP DEVICE AND CLIP UNIT

(71) Applicant: OLYMPUS MEDICAL SYSTEMS CORP., Tokyo (JP)

(72) Inventor: Shogo Shindo, Hachioji-shi (JP)

(21) Appl. No.: 19/420,105

(22) Filed: Dec. 15, 2025

Related U.S. Application Data
(63) Continuation of application No. PCT/JP2024/021752, filed on Jun. 14, 2024.
(60) Provisional application No. 63/508,620, filed on Jun. 16, 2023.

(51) Int. Cl. A61B 17/00

(52) U.S. Cl. CPC A61B 17/0057; A61B 17/00234; A61B 2017/00296; A61B 2017/00477; A61B 2017/00584; A61B 2017/00623

(57) ABSTRACT

A clip device includes a clip unit having arms that open and close in a front direction and a holder that supports the arms to be pivotable in opening and closing directions, a sheath that is detachably connected to the clip unit, and a wire that passes through the sheath and is detachably connected to the arms, in which the arms have a first arm and a second arm, and the arms are able to transition between a first mode in which the first arm and the second arm pivot independently and a second mode in which the first arm, which pivots in an opening direction of the opening and closing directions, causes the second arm to pivot in a closing direction of the opening and closing directions.

### CLAIM FOR PRIORITY

This application is a continuation of and claims the benefit of priority of International Application No. PCT/JP2024/021752, filed on Jun. 14, 2024, and published as WO2024/257871 on Dec. 19, 2024, which claims priority to U.S. Provisional Application No. 63/508,620, filed Jun. 16, 2023; the benefit of priority of each of which is hereby claimed herein, and which applications and publication are hereby incorporated herein by reference in their entireties.

### BACKGROUND OF THE INVENTION

### Field of the Invention

The present invention relates to a clip device and a clip unit. Priority is claimed on U.S. Provisional Application No. 63/508,620, filed Jun. 16, 2023, the contents of which are incorporated herein by reference.

### Description of Related Art

In endoscopic treatments, medical instruments such as clip devices are used to treat biological tissue. These medical instruments are introduced to a treatment position using an applicator (introduction device) that can pass through a channel of an endoscope.

United States Patent Application, Publication No. 2021/030425 describes a clipping device for treating biological tissue. In the clipping device described in United States Patent Application, Publication No. 2021/030425, a control wire that controls a clip has a bending portion. Bending of the bending portion of the control wire allows the clip connected to the control wire to move significantly and come into contact with the tissue.

### SUMMARY OF THE INVENTION

A clip device according to a first aspect of the present invention includes a clip unit having arms that open and close in a front direction and a holder that supports the arms to be pivotable in opening and closing directions, a sheath that is detachably connected to the clip unit, and a wire that passes through the sheath and is detachably connected to the arms, in which the arms have a first arm and a second arm, and the arms are able to transition between a first mode in which the first arm and the second arm pivot independently and a second mode in which the first arm, which pivots in an opening direction of the opening and closing directions, causes the second arm to pivot in a closing direction of the opening and closing directions.

A clip device according to a second aspect of the present invention includes a clip unit having arms that open and close in a front direction and a holder that supports the arms to be pivotable in opening and closing directions, a sheath that is detachably connected to the clip unit and extends in a longitudinal axis direction, and a wire that passes through the sheath and is detachably connected to the arms, in which the arms are able to transition between a first mode in which the front direction is aligned with the longitudinal axis direction and a second mode in which the front direction intersects with the longitudinal axis direction.

A clip unit according to a third aspect of the present invention is a clip unit loaded into a sheath of an applicator, and includes arms that open and close toward a front direction, and a holder that supports the arm to be pivotable in opening and closing directions, in which the arms have a first arm and a second arm, the arms are able to transition between a first mode in which the first arm and the second arm pivot independently, and a second mode in which the first arm, which pivots in an opening direction of the opening and closing directions, causes the second arm to pivot in a closing direction of the opening and closing directions.

### BRIEF DESCRIPTION OF THE DRAWINGS

FIG. 1 is a diagram which shows a clip device according to a first embodiment.

FIG. 2 is a cross-sectional view of a clip unit loaded into an applicator.

FIG. 3 is a cross-sectional view of the clip unit loaded into the applicator.

FIG. 4 is a diagram which shows the clip unit with a pair of arms in an open mode.

FIG. 5 is a diagram which shows a transition of the mode of the pair of arms.

FIG. 6 is a diagram which shows a clip locked in a closed mode.

FIG. 7 is a diagram which shows the clip locked in the closed mode.

FIG. 8 is a diagram which shows the clip device with the sheath and holder of the applicator separated.

FIG. 9 is a diagram which shows the clip device with the sheath and holder of the applicator separated.

FIG. 10 is a diagram which shows the clip unit treating a tissue using a tangential approach.

FIG. 11 is a diagram which shows the clip unit with the pair of arms swinging.

FIG. 12 is a diagram which shows a lock step performed by the clip unit.

FIG. 13 is a diagram which shows a separation step performed by the clip unit.

FIG. 14 is a diagram which shows a modified example of a clip of the clip unit.

FIG. 15 is a diagram which shows the modified example of the clip.

FIG. 16 is a diagram which shows the modified example of the clip.

FIG. 17 is a diagram which shows the modified example of the clip.

### DETAILED DESCRIPTION OF THE INVENTION

### First Embodiment

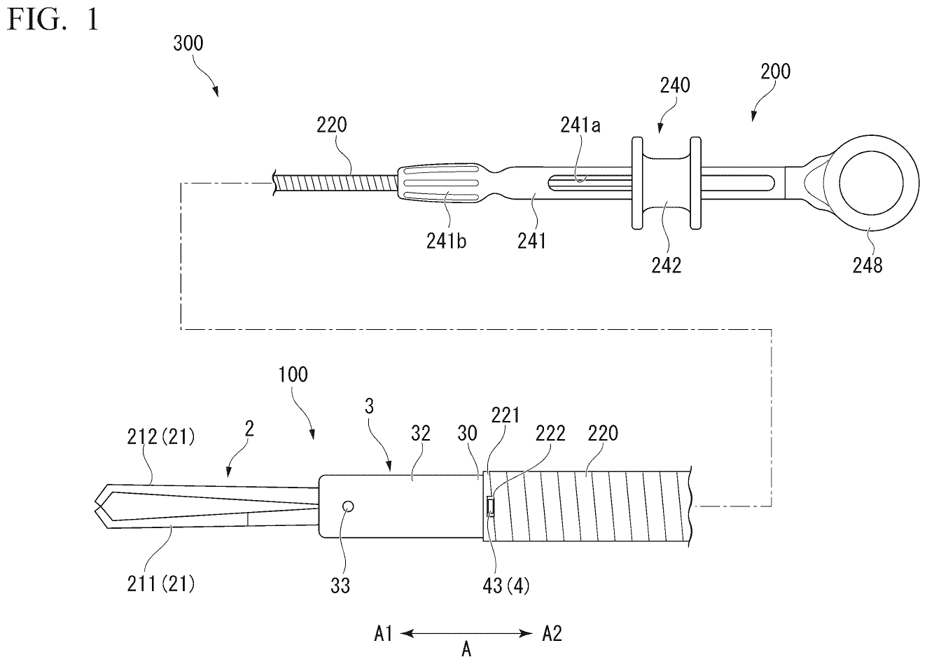

A first embodiment of the present invention will be described with reference to FIGS. 1 to 13.

FIG. 1 shows a diagram which shows a clip device 300 according to the present embodiment.

The clip device (clip system, endoscopic treatment tool) 300 includes a clip unit and an applicator 200. The clip unit 100 is loaded into the applicator 200, which can pass through a channel of an endoscope, and introduced to a treatment position.

The clip device 300 may be capable of reloading the clip unit 100 onto the applicator 200. In this case, a user can remove a used clip unit 100 from the applicator and reload an unused clip unit 100 into the applicator 200.

In the following description, a side of the clip unit 100 in a longitudinal direction A of the clip device 300 will be referred to as a distal end side (distal side) A1 of the clip device 300, and a side of the operating portion 240 of the applicator 200 will be referred to as a proximal end side (proximal side) A2 of the clip device 300.

### [Applicator 200]

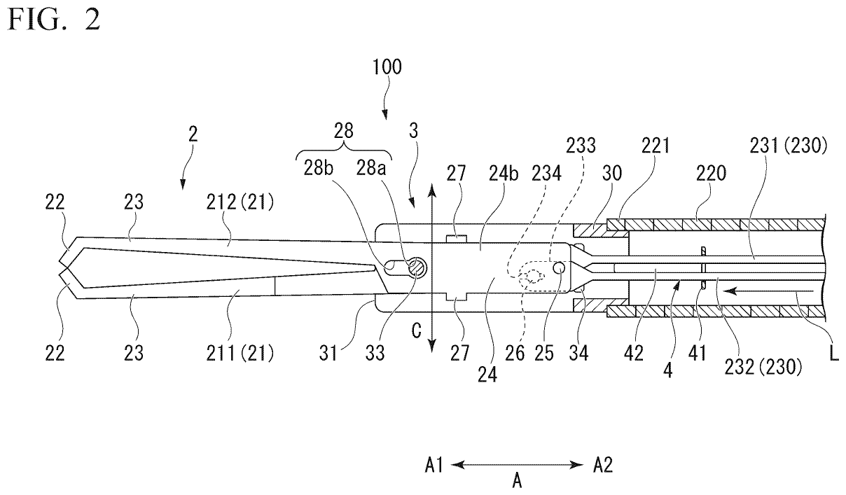

FIG. 2 is a cross-sectional view of the clip unit 100 loaded into the applicator 200.

The applicator (clip unit introduction device) 200 includes a sheath 220, a control wire 230, and an operating portion 240. The applicator 200 passes through, for example, a treatment tool insertion channel of an endoscope and is used in combination with the endoscope. For this reason, the sheath 220 is formed to be much longer than a length of the treatment tool insertion channel of the endoscope. The sheath 220 is flexible and bends to match bending of the insertion portion of the endoscope.

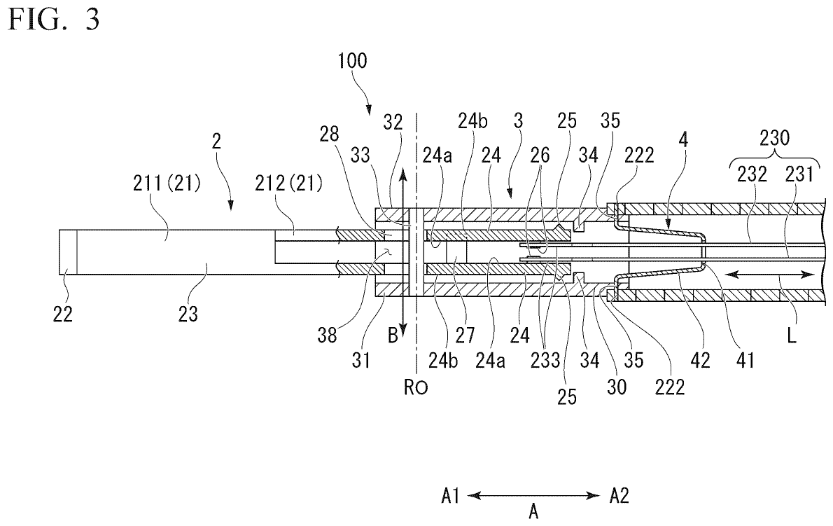

FIG. 3 is a cross-sectional view of the clip unit 100 loaded into the applicator 200.

The sheath 220 is a coil sheath. A distal end portion 221 of the sheath 220 is provided with an engaging hole 222 with which a connecting member 4 of the clip unit 100 engages. The engaging holes 222 are provided on both sides of a central axis in a longitudinal axis direction L of the sheath 220.

The control wire (power transmission member) 230 passes through the sheath 220 and is attached to the clip unit 100 and the operating portion 240. As shown in FIG. 3, the control wire 230 can protrude from the distal end portion 221 of the sheath 220 to the distal end side A1. The control wire 230 moves the distal end portion 221 back and forth in the longitudinal axis direction L of the sheath 220 to transmit power to the clip 2 of the clip unit 100.

A distal end of the control wire 230 is provided with a connecting portion 233 that is detachably connected to a connecting portion 26 of the clip 2, which will be described below. An outer diameter of the connecting portion 233 is larger than an outer diameter of the control wire 230. A distal end of the connecting portion 233 is provided with a slit 234 into which the connecting portion 26 of the clip 2, which will be described below, is fitted so as to be relatively movable.

The control wire 230 has a first control wire 231 and a second control wire 232. The first control wire 231 and the second control wire 232 run parallel to each other in an internal space of the sheath 220.

The operating portion 240 includes an operating portion main body 241, a slider 242, and a thumb ring 248. The operating portion main body 241, slider 242, and thumb ring 248 are injection molded from, for example, a resin material. The operating portion main body 241 includes a slit portion 241a and a rotating grip 241b on the distal end side. The slit portion 241a supports the slider 242 so that it can move back and forth.

The slider 242 is attached to the operating portion main body 241 so that it can move back and forth in a longitudinal axis direction of the operating portion main body, and a base end of the control wire 230 (first control wire 231 and second control wire 232) is attached thereto. As the slider 242 moves back and forth along the operating portion main body 241, the control wire 230 (first control wire 231 and second control wire 232) moves back and forth relative to the sheath 220.

The thumb ring 248 is attached to the base end of the operating portion main body 241 so that it is pivotable about a longitudinal axis of the operating portion main body 241.

### [Clip Unit 100]

The clip unit 100 is loaded into the distal end portion 221 of the sheath 220 of the applicator 200. The clip unit 100 includes a clip 2, a holder 3, and a connecting member 4.

The clip (clip arm) 2 has a pair of arms 21. Note that the clip (clip arm) 2 may have auxiliary arms other than the pair of arms 21.

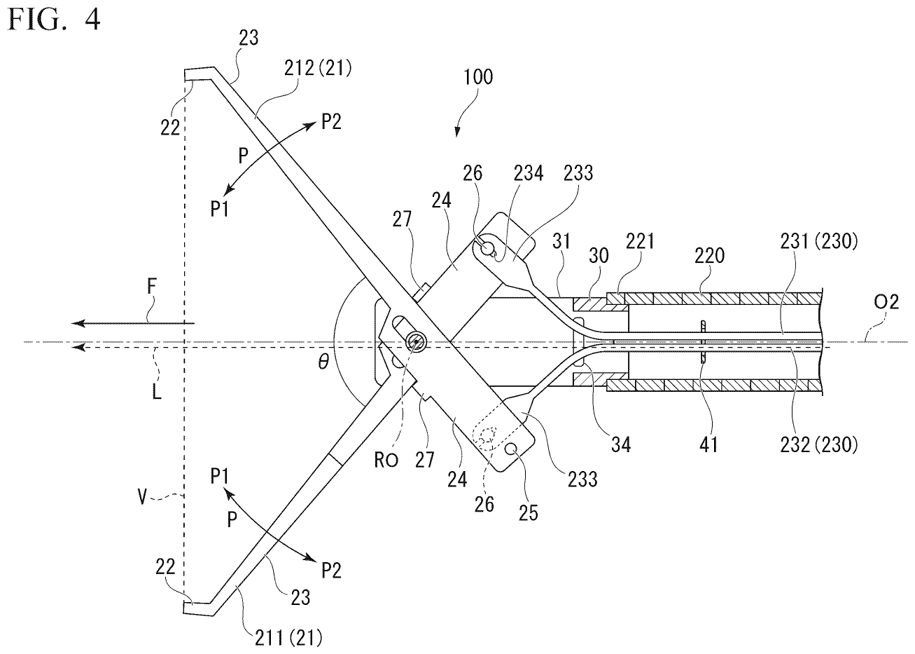

FIG. 4 is a diagram which shows the clip unit 100 with the pair of arms 21 in an open mode.

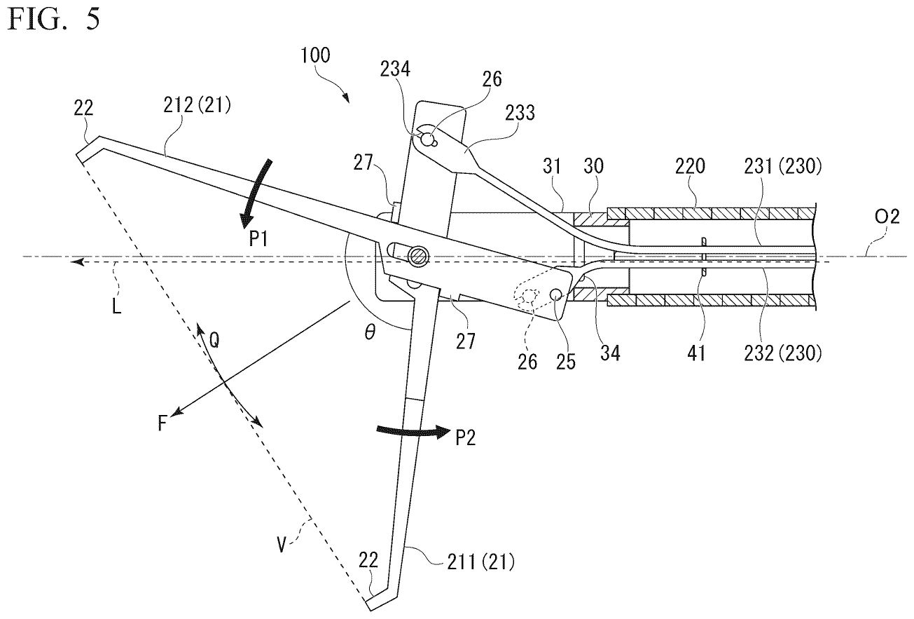

The pair of arms 21 can be opened and closed in a front direction F, and can transition between the open mode (open state) and a closed mode (closed state). The pair of arms 21 can swing in a swing direction Q. The front direction F changes when the pair of arms 21 swing (refer to FIG. 5).

The pair of arms 21 include a first arm 211 and a second arm 212. The first arm 211 and the second arm 212 are supported by the holder 3 so as to be pivotable in opening and closing directions P about a pivot axis RO along a vertical direction B which is substantially perpendicular to the longitudinal direction A. The first arm 211 and the second arm 212 are separately supported by the holder 3.

A direction in which one arm approaches the other arm in the opening and closing directions P is referred to as a “closing direction P1.” A direction in which one arm moves away from the other arm in the opening and closing directions P is referred to as an “opening direction P2.” A direction in which the pair of arms 21 face each other is referred to as an “inside,” and a side opposite the inside is referred to as an “outside.”

The first arm 211 and the second arm 212 each have, from the distal end side A1 to the proximal end side A2, a claw 22, a grip portion 23, a main body portion 24, and an engaging portion 25. The main body portion 24 has a connecting portion 26 and a link portion 27.

The claw 22 is formed by bending the distal ends of the first arm 211 and the second arm 212 inward.

As shown in FIG. 2, the grip portion 23 is formed in a flat plate shape and is a portion that grips tissue. The grip portion 23 of the first arm 211 and the grip portion 23 of the second arm 212 are provided so that their flat surfaces face each other.

As shown in FIG. 3, the main body portion 24 is formed in a flat plate shape and is supported by a pin 33 of the holder 3 to be pivotable in the opening and closing directions P. Specifically, a through-hole 28 formed in an inner surface 24a of the main body portion 24 is fitted into the pin 33 of the holder 3, so that the main body portion 24 is supported to be pivotable with respect to the holder 3. The main body portion 24 is provided so that a plate thickness direction thereof substantially coincides with the vertical direction B.

The engaging portion 25 is a portion that can engage with an engaging portion 34 of the holder 3. The engaging portion 25 is a convex portion that protrudes outward from an outer surface 24b of the main body portion 24.

As shown in FIG. 2, the through-hole 28 has a first through-hole 28a that is circular when viewed in the vertical direction B, and a second through-hole 28b that is oval when viewed from in the vertical direction B. The second through-hole 28b is continuous with the first through-hole 28a and extends from the first through-hole 28a to the distal end side A1.

The connecting portion 26 is a portion that is detachably connected to the slit 234 of the connecting portion 233 of the control wire 230. The connecting portion 26 is a convex portion that protrudes inward from the inner surface 24a of the main body portion 24. When the control wire 230 is pulled with a force greater than a predetermined value, the connecting portion 26 comes out of the slit 234, and the control wire 230 and the connecting portion 26 are separated.

The link portion 27 engages with the other arm when the pair of arms 21 are in the swing mode, which will be described below. As shown in FIG. 3, the link portion 27 is provided at an end of the main body portion 24 in a width direction C, which is substantially perpendicular to the longitudinal direction A and the vertical direction B. The link portion 27 extends inward from the main body portion 24. The claw 22 and the link portion 27 are provided on opposite sides of the pivot axis RO.

FIG. 5 is a diagram which shows a transition of a mode of the pair of arms 21.

The pair of arms 21 can transition between a normal mode (first mode) shown in FIG. 4 and a swing mode (second mode) shown in FIG. 5.

The normal mode (first mode) is an operation mode in which the first arm 211 and the second arm 212 pivot independently. When the pair of arms 21 are in the normal mode, as shown in FIG. 4, the front direction F of the pair of arms 21 is aligned along the longitudinal axis direction L of the sheath 220. That is, the normal mode is a mode in which an imaginary line segment V is orthogonal to a central axis line O2 in the longitudinal axis direction L of the sheath 220. Here, the imaginary line segment V is a line segment connecting distal ends of the pair of arms 21 in the open mode. In addition, the normal mode is a mode in which, in the pair of arms 21 in the open mode, a distance between the distal end of the first arm 211 and the central axis line O2 of the sheath 220 is substantially equal to a distance between the distal end of the second arm 212 and the central axis line O2 of the sheath 220.

Note that when the pair of arms 21 are in the normal mode, the front direction F of the pair of arms 21 does not necessarily have to be aligned along the longitudinal axis direction L of the sheath 220. The first arm 211 and the second arm 212 may pivot independently so that the front direction F of the pair of arms 21 is not aligned along the longitudinal axis direction L of the sheath 220.

The swing mode (second mode) is an operation mode in which one arm (one of the first arm 211 and the second arm 212), which pivots in the opening direction P2 of the opening and closing directions P, causes the other arm (the other of the first arm 211 and the second arm 212) to pivot in the closing direction P1 of the opening and closing directions P. When the pair of arms 21 are in the swing mode, as shown in FIG. 5, the front direction F of the pair of arms 21 is aligned in a direction intersecting with the longitudinal axis direction L of the sheath 220. In other words, the swing mode of the clip unit 100 is a mode in which the imaginary line segment V intersects with the central axis line O2 of the sheath 220. Moreover, the swing mode is a mode in which, in the pair of arms 21 in the open mode, the distance between the distal end of the first arm 211 and the central axis line O2 of the sheath 220 is smaller or larger than the distance between the distal end of the second arm 212 and the central axis line O2 of the sheath 220.

An angle by which the first arm 211 and the second arm 212 open toward the front direction F is defined as an opening and closing angle θ. When the opening and closing angle θ is less than a predetermined angle, the pair of arms 21 is in the normal mode (first mode). When the opening and closing angle θ is the predetermined angle, the pair of arms 21 is in the swing mode (second mode).

When the opening and closing angle θ reaches the predetermined angle, the link portion 27 of the first arm 211, which pivots in the opening direction P2, engages with the second arm 212. In addition, the first arm 211 engages with the link portion 27 of the second arm 212. Furthermore, when the first arm 211 pivots in the opening direction P2, the link portion 27 of the first arm 211 causes the second arm 212 to pivot in the closing direction P1 of the opening and closing directions P. The first arm 211 also causes the link portion 27 of the second arm 212 to pivot in the closing direction P1 of the opening and closing directions P. As a result, the pair of arms 21 swing in the swing direction Q. The swing direction (pivot direction) Q substantially coincides with the opening and closing directions P of the pair of arms 21. In other words, a plane in which the pair of arms 21 swing (a plane through which the pair of swinging arms 21 pass) and a plane in which the pair of arms 21 open and close (a plane through which the pair of opening and closing arms 21 pass) substantially coincide with each other.

When the opening and closing angle θ is the predetermined angle, the second arm 212, which pivots in the opening direction P2 of the opening direction and closing direction P, similarly causes the first arm 211 to pivot in the closing direction P1 of the opening direction and closing direction P. As a result, the pair of arms 21 swing in the swing direction Q.

For example, when the first arm 211, which has pivoted in the opening direction P2, pivots in the closing direction P1, the opening and closing angle θ becomes less than the predetermined angle. When the opening and closing angle θ becomes less than the predetermined angle, the pair of arms 21 return to the normal mode (first mode).

The holder 3 supports the clip 2 to be pivotable in the opening and closing directions P about the pivot axis RO along the vertical direction B, which is substantially perpendicular to the longitudinal direction A. The holder 3 has an internal space 38 in which the main body portion 24 of the clip 2 is stored.

As shown in FIG. 3, the holder 3 has a proximal tubular portion 30, a first support portion 31, a second support portion 32, a pin 33, and an engaging portion 34.

The proximal tubular portion 30 is a rigid member formed of metal or the like, and is formed in an annular shape. The proximal tubular portion 30 has an engaging hole 35 with which the connecting member 4 engages. The engaging holes 35 are provided on both sides of the central axis, which is along the longitudinal axis direction L of the sheath 220.

The first support portion 31 and the second support portion 32 are formed in a plate shape and extend from the proximal tubular portion 30 to the distal end side A1. The first support portion 31 and the second support portion 32 are provided opposite each other in the vertical direction B. The pair of arms 21 are disposed between the first support portion 31 and the second support portion 32.

The pin 33 is fixed to the first support portion 31 and the second support portion 32. The pin 33 passes through the through-hole 28 in the main body portion 24 of the pair of arms 21. The pin 33 is formed in a cylindrical shape. A central axis of the pin 33 is the pivot axis RO of the pair of arms 21.

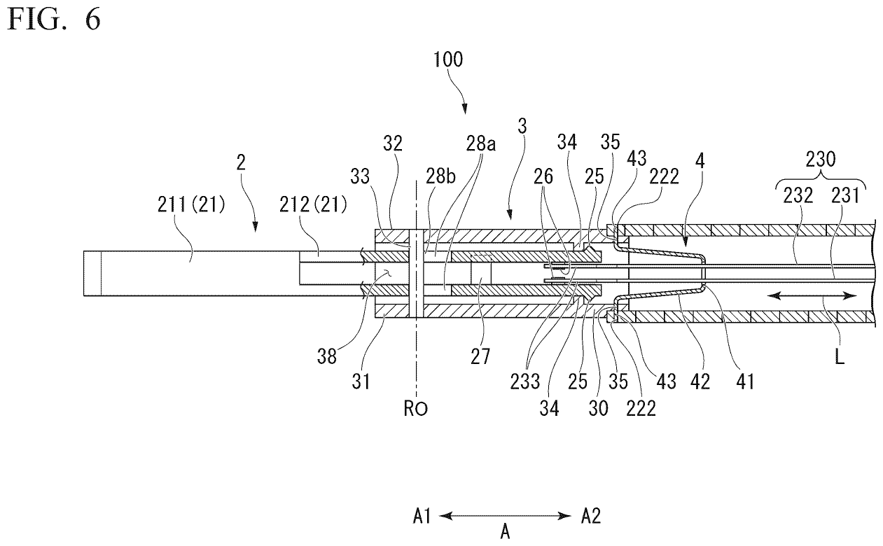

FIG. 6 is a diagram which shows the clip 2 locked in the closed mode.

The engaging portion 34 is a portion that can engage with the engaging portions 25 of the pair of arms 21. The engaging portion 34 is a convex portion that protrudes from an inner circumferential surface of the proximal tubular portion 30 toward the pair of arms 21. As shown in FIG. 6, when the pair of arms 21 move to the proximal end side A2 and the engaging portion 25 moves closer to the proximal end side A2 than the engaging portion 34, the engaging portion 25 cannot move closer to the distal end side A1 than the engaging portion 34.

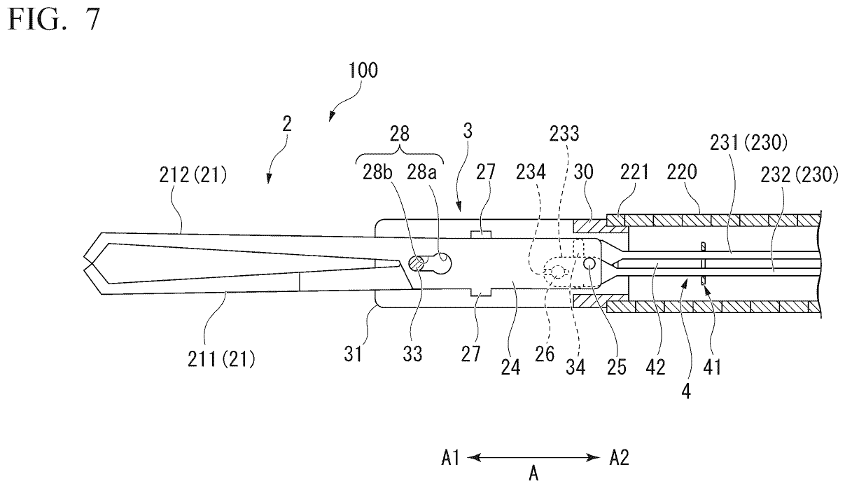

FIG. 7 is a diagram which shows the clip 2 locked in the closed mode.

As the clip 2 moves to the proximal end side A1 so that a position of the pin 33 moves from the first through-hole 28a to the second through-hole 28b, the engaging portion 25 moves closer to the proximal end side A2 than the engaging portion 34. By pulling the clip 2 to the proximal end side A2, the clip 2 transition to the closed mode, and the position of the pin 33 moves from the first through-hole 28a to the second through-hole 28b. As a result, the engaging portion 25 moves closer to the proximal end side A2 than the engaging portion 34, and the clip 2 are locked in the closed mode.

The engaging portion 25 and the engaging portion 34 of the holder 3 together constitute a locking mechanism that locks the clip 2 in the closed mode. Once the pair of arms 21 are locked in the closed mode by the locking mechanism, the pair of arms 21 cannot return to the open mode. The locking mechanism is not limited to a combination of the engaging portion 25 of the clip 2 and the engaging portion 34 of the holder 3, and may also be another constituent.

The connecting member 4 detachably connects the sheath 220 and the holder 3. The connecting member 4 has an annular portion 41 and a pair of support arms 42.

The annular portion 41 is formed in a ring shape. The control wire 230 (first control wire 231 and second control wire 232) passes through the annular portion 41.

The pair of support arms 42 are elastic and are provided on both sides of the central axis, which is along the longitudinal axis direction L of the sheath 220. The distal end portions 43 of the pair of support arms 42 pass through the engaging holes 222 of the sheath 220 and the engaging holes 35 of the holder 3. The distal end portions 43 pass through the engaging holes 222 of the sheath 220 and the engaging holes 35 of the holder 3, thereby connecting the sheath 220 and the holder 3.

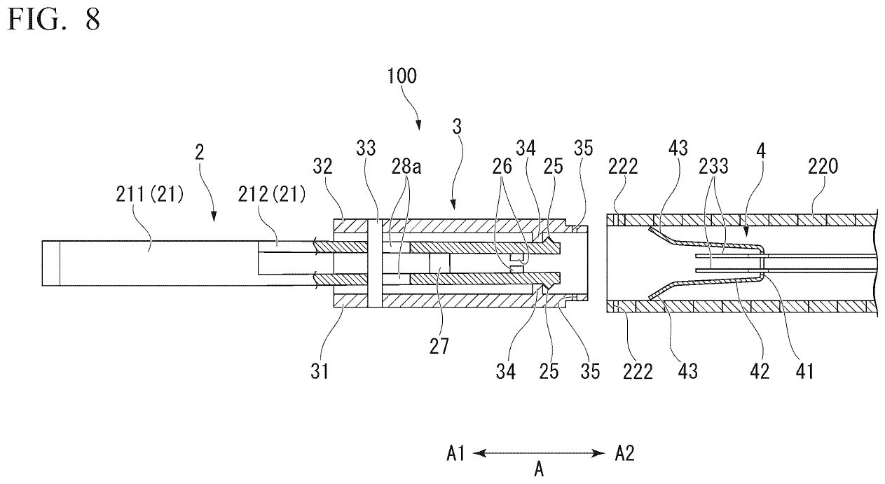

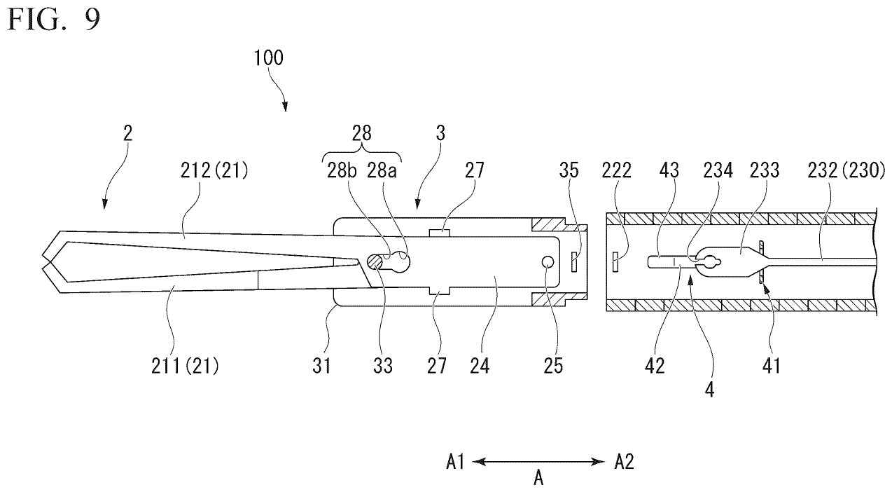

FIGS. 8 and 9 are diagrams which show the clip device 300 in which the sheath 220 and the holder 3 have been separated. After the clip 2 is locked in the closed mode, when the control wire 230 is further pulled toward the proximal end side A2, the slit 234 of the connecting portion 233 is disengaged from the connecting portion 26 of the clip 2. As the control wire 230 is further pulled toward the proximal end side A2, the connecting portion 233 engages with the annular portion 41, thereby moving the connecting member 4 to the proximal end side A2. A distal end portion 43 of the connecting member 4 disengages from the engaging hole 222 of the sheath 220 and the engaging hole 35 of the holder 3. As a result, the sheath 220 and the holder 3 are separated.

### [Operation and Function of Clip Unit 100]

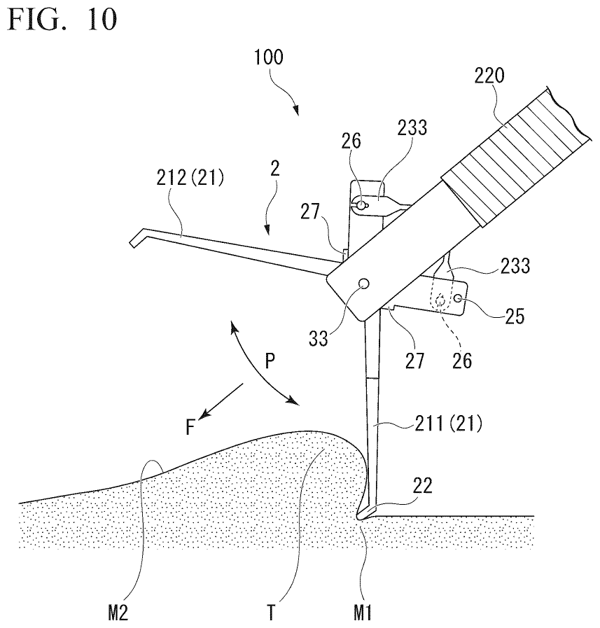

Next, an operation and a function of the clip unit 100 will be described with reference to FIGS. 10 to 13.

### <Approach Step>

A surgeon introduces the clip unit 100, loaded into the applicator 200, into a body via the channel of the endoscope. The surgeon moves the endoscope or sheath 220 to bring clip 2 of the clip unit 100 closer to tissue T, which is a treatment area.

There are at least two methods for bringing the clip unit 100 close to the treatment area: a frontal approach and a tangential approach. In the frontal approach, the surgeon manipulates the endoscope or sheath 220 to dispose the clip unit 100 so that the longitudinal direction A thereof is substantially perpendicular to a surface of the treatment area. In the tangential approach, the surgeon manipulates the endoscope or sheath 220 to dispose the clip unit 100 so that the longitudinal direction A thereof is aligned with the surface of the treatment area.

### <Tangential Approach>

FIG. 10 is a diagram which shows a clip unit 100 that treats tissue T using a tangential approach. The surgeon brings the clip 2 in the open mode, which is a normal mode, to the tissue T that is a treatment area using the tangential approach. The surgeon hooks the claw 22 of the first arm 211 onto a mucosa on the endoscope side (a near side) of the first mucosa M1 and second mucosa M2, which are located on both sides of a center of the tissue T (first mucosa M1 in FIG. 10).

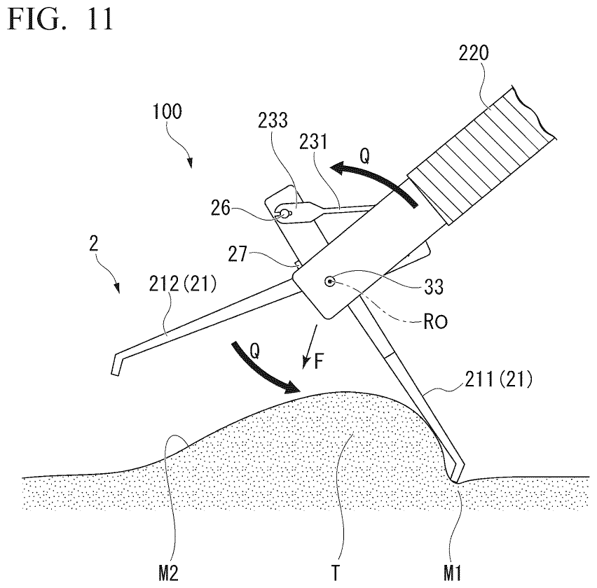

FIG. 11 shows the clip unit 100 with the pair of arms 21 swung. The surgeon further brings the clip unit 100 with the claw 22 hooked onto the first mucosa M1 closer to the tissue T, which is the treatment area. Specifically, the surgeon advances the applicator 200 relative to the channel of the endoscope. Alternatively, the surgeon advances the endoscope, thereby pressing the clip unit 100 further against the tissue T. The link portion 27 of the first arm 211, which pivots in the opening direction P2 of the opening and closing directions P, causes the second arm 212 to pivot in the closing direction P1 of the opening and closing directions P. Furthermore, the first arm 211 causes the link portion 27 of the second arm 212 to pivot in the closing direction P1 of the opening and closing directions P. As a result, as shown in FIG. 11, the pair of arms 21 swing in the swing direction Q about the pivot axis RO. The clip 2 transitions to the swing mode. When the second arm 212 contacts the second mucosa M2, the pair of arms 21 are disposed so that the front direction F faces a front of the tissue T, which is the treatment area. The first mucosa M1 and the second mucosa M2 are gripped by the pair of arms 21.

The surgeon may advance the first control wire 231 to cause the first arm 211 to pivot in the opening direction P2, thereby transitioning the clip 2 from the normal mode to the swing mode.

### <Pulling Step>

The user retracts the control wire 230 by retracting the slider 242 along the operating portion main body 241. The connecting member 4 connected to the control wire 230 pulls the clip 2. As the clip 2 is pulled toward the proximal end side A2, the pair of arms 21 are gradually closed.

### <Lock Step>

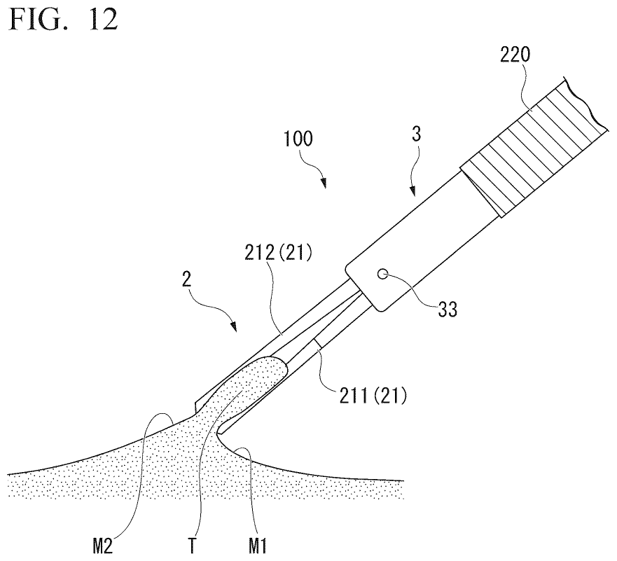

FIG. 12 is a diagram which shows a lock step performed by the clip unit 100.

The surgeon further retracts the control wire 230 by retracting the slider 242 along the operating portion main body 241. When the clip 2 is retracted to a desired position in the holder 3, the engaging portion 25 of the clip 2 engages with the engaging portion 34 of the holder 3, thereby locking the pair of arms 21 into a closed state. Once the pair of arms 21 are locked in the closed state, the pair of arms 21 cannot return to the open state.

### <Separation Step>

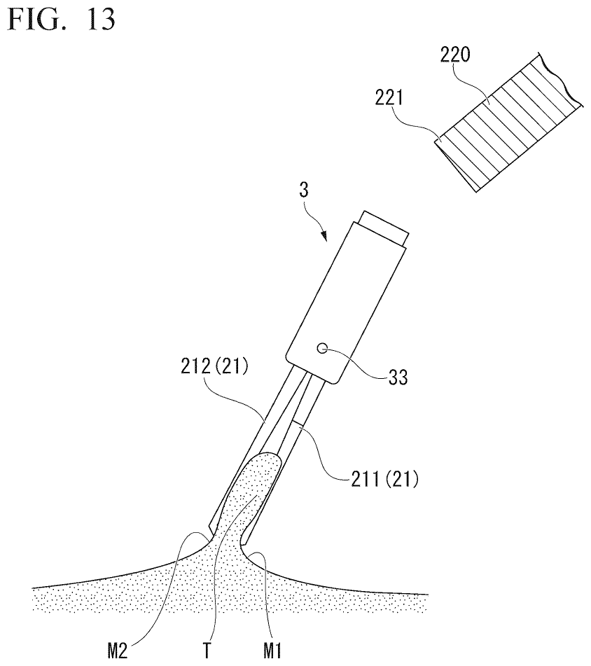

FIG. 13 shows a separation step performed by the clip unit 100.

The surgeon further pulls the clip 2. The distal end portion 43 of the connecting member 4 disengages from the engaging hole 222 of the sheath 220 and the engaging hole 35 of the holder 3. As a result, the sheath 220 and the holder 3 are separated. The surgeon retracts the sheath 220, thereby leaving the clip 2 and the holder 3 in the body with the first mucosa M1 and the second mucosa M2 ligated.

The clip unit 100 of the present embodiment can suitably grasp the tissue T in a lumen. Even when the tissue T is treated using the tangential approach, the clip 2 can be swung to dispose the pair of arms 21 to face the front of the tissue T, which is the treatment area. Because one arm of the clip unit 100 swings in conjunction with the other arm, it is easy to dispose the clip unit 100 to face the front of the tissue T, even in a narrow lumen.

A first embodiment of the present invention has been described in detail above with reference to the drawings. However, a specific configuration is not limited to the present embodiment, and design modifications that do not deviate from the gist of the present invention are also included. Furthermore, components shown in an embodiment and modified example can be combined as appropriate.

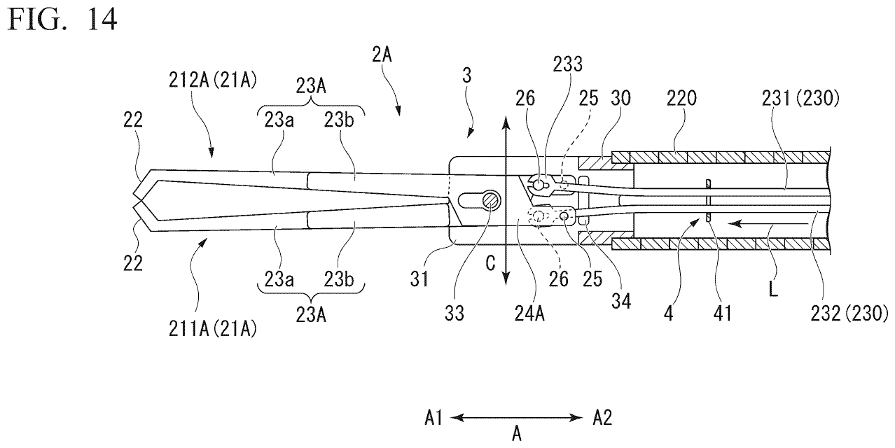

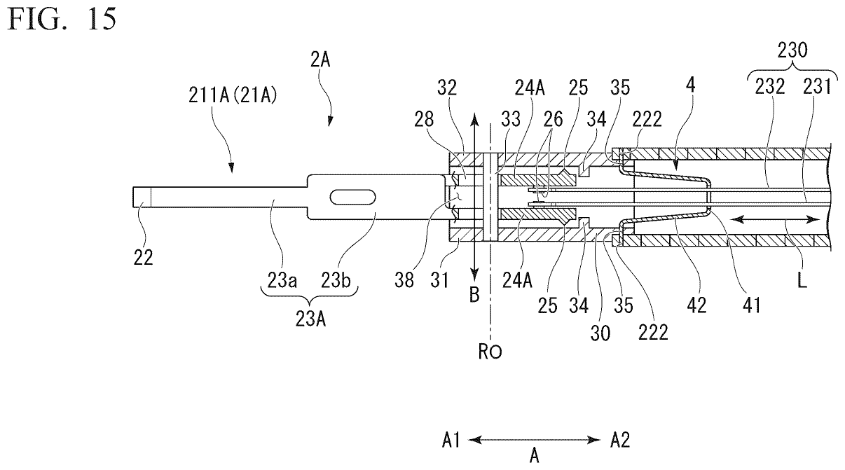

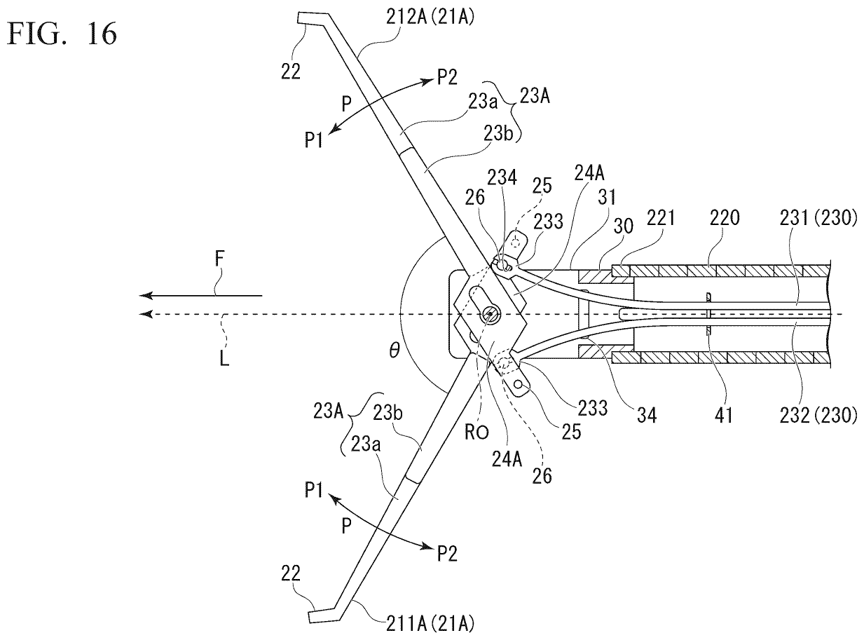

FIGS. 14 to 17 are diagrams which show a clip 2A, a modified example of the clip 2.

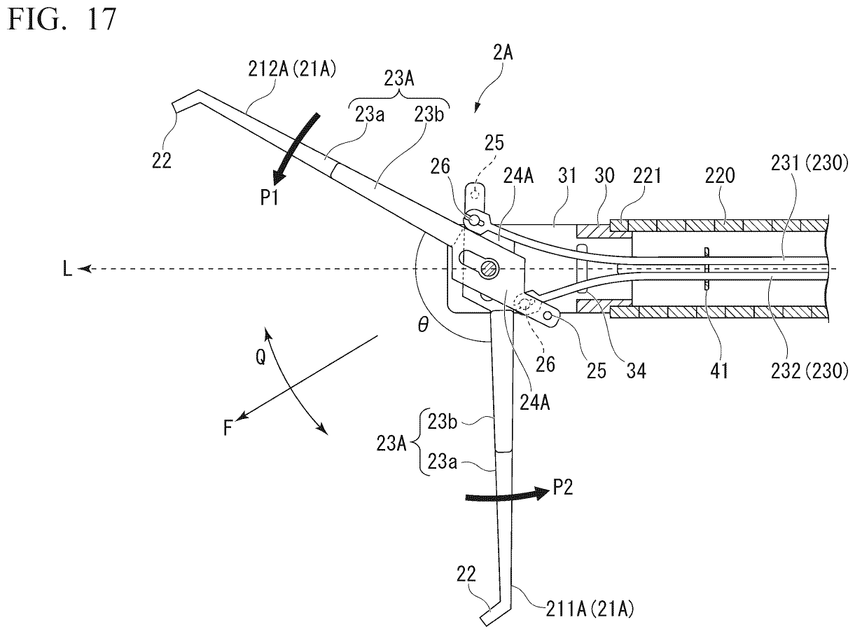

The clip 2A has a pair of arms 21A. The pair of arms 21A can be opened and closed in the front direction F, and can transition between the open mode (open state) and the closed mode (closed state). The pair of arms 21A can swing in the swing direction Q. The front direction F changes when the pair of arms 21A swing (refer to FIG. 17).

The pair of arms 21A has a first arm 211A and a second arm 212A. The first arm 211A and the second arm 212A are supported by the holder 3 to be pivotable in the opening and closing directions P about the pivot axis RO along the vertical direction B. The first arm 211A and the second arm 212A are separately supported by the holder 3.

The first arm 211A and the second arm 212A have, from the distal end side A1 to the proximal end side A2, the claw 22, a grip portion 23A, a main body portion 24A, and an engaging portion 25. The main body portion 24A has a connecting portion 26. The main body portion 24A does not have a link portion 27.

The grip portion 23A is formed in a flat plate shape and is the part that grips tissue. The grip portion 23A of the first arm 211A and the grip portion 23A of the second arm 212A are arranged so that their flat surfaces face each other.

The grip portion 23A has a distal end grip portion 23a provided on the distal end side A1 and a base grip portion 23b provided on the proximal end side A2. A width of the base grip portion 23b in the vertical direction B is wider than a width of the distal end grip portion 23a in the vertical direction B. The width of the base grip portion 23b in the vertical direction B is wider than the width of the main body portion 24A in the vertical direction B.

Similar to the main body portion 24 in the embodiment described above, the main body portion 24A is supported by the pin 33 of the holder 3 to be pivotable in the opening and closing directions P.

When the opening and closing angle θ reaches a predetermined angle, the main body portion (link portion) 24A of the first arm 211A, which pivots in the opening direction P2, engages with the base grip portion 23b of the second arm 212A. The base grip portion 23b of the first arm 211A also engages with the main body portion (link portion) 24A of the second arm 212A. Furthermore, when the first arm 211A pivots in the opening direction P2, the main body portion (link portion) 24A of the first arm 211A causes the base grip portion 23b of the second arm 212A to pivot in the closing direction P1 of the opening and closing directions P. Furthermore, the base grip portion 23b of the first arm 211A causes the main body portion (link portion) 24A of the second arm 212A to pivot in the closing direction P1 of the opening and closing directions P. As a result, the pair of arms 21A swing in the swing direction Q.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A clip device comprising:
a clip unit having arms that open and close and a holder that supports the arms to be pivotable in opening and closing directions;
a sheath that is detachably connected to the clip unit; and
a wire that passes through the sheath and is detachably connected to the arms;
wherein the arms have a first arm and a second arm, and
the arms are able to transition between a first mode in which the first arm and the second arm pivot independently and a second mode in which the first arm, which pivots in an opening direction of the opening and closing directions, causes the second arm to pivot in a closing direction of the opening and closing directions.

2. The clip device according to claim 1,
wherein an angle by which the first arm and the second arm open is set as an opening and closing angle,
the arms are in the first mode when the opening and closing angle is less than a predetermined angle, and
the arms are in the second mode when the opening and closing angle is equal to the predetermined angle.

3. The clip device according to claim 2,
wherein the first arm has a link portion that engages with the second arm when the arms are in the second mode.

4. The clip device according to claim 3,
wherein the first arm is supported by the holder to be pivotable about a pivot axis, and
a distal end of the first arm and the link portion of the first arm are provided on opposite sides of the pivot axis.

5. The clip device according to claim 3,
wherein the second arm, which pivots in the opening direction of the opening and closing directions, is caused to pivot in the closing direction of the opening and closing directions by the first arm in the second mode, and
the second arm has a link portion that engages with the first arm when the arms are in the second mode.

6. The clip device according to claim 5,
wherein the second arm is supported by the holder to be pivotable about a pivot axis, and
a distal end of the second arm and the link portion of the second arm are provided on opposite sides of the pivot axis.

7. The clip device according to claim 1, further comprising:
a connecting member configured to detachably connect the sheath and the holder,
wherein the connecting member separates the sheath and the holder when the wire is pulled.

8. The clip device according to claim 1,
wherein the clip unit has a locking mechanism that locks the arms in a closed mode.

9. The clip device according to claim 8,
wherein the locking mechanism has an engaged portion provided on the holder and an engaging portion provided on the arms, and
when the arms are pulled by the wire, the engaging portion engages with the engaged portion, thereby locking the arms in the closed mode.

10. A clip device comprising:
a clip unit having arms that open and close and a holder that supports the arms to be pivotable in opening and closing directions;
a sheath that is detachably connected to the clip unit and extends in a longitudinal axis direction; and
a wire that passes through the sheath and is detachably connected to the arms,
wherein the arms are able to transition between a first mode in which the arms open and close about a central axis of the sheath and a second mode in which the arms open and close about the central axis.

11. The clip device according to claim 10,
wherein the arm has a first arm and a second arm,
an angle by which the first arm and the second arm open toward the front direction is set as an opening and closing angle,
the arms are in the first mode when the opening and closing angle is less than a predetermined angle, and
the arms are in the second mode when the opening and closing angle is equal to the predetermined angle.

12. The clip device according to claim 11,
wherein the first arm has a link portion that engages with the second arm when the arms are in the second mode.

13. The clip device according to claim 12,
wherein the first arm is supported by the holder to be pivotable about a pivot axis, and
a distal end of the first arm and the link portion of the first arm are provided on opposite sides of the pivot axis.

14. The clip device according to claim 12,
wherein the second arm has a link portion that engages with the first arm when the arms are in the second mode.

15. The clip device according to claim 14,
wherein the second arm is supported by the holder to be pivotable about a pivot axis, and
a distal end of the second arm and the link portion of the second arm are provided on opposite sides of the pivot axis.

16. The clip device according to claim 10, further comprising:
a connecting member that detachably connects the sheath and the holder,
wherein the connecting member separates the sheath and the holder when the wire is pulled.

17. The clip device according to claim 10,
wherein the clip unit has a locking mechanism that locks the arms in a closed mode.

18. The clip device according to claim 17,
wherein the locking mechanism includes an engaged portion provided on the holder and an engaging portion provided on the arm, and
when the arms are pulled by the wire, the engaging portion engages with the engaged portion, thereby locking the arms in the closed mode.

19. A clip unit loaded into a sheath of an applicator comprising:
arms that open and close; and
a holder that supports the arm to be pivotable in opening and closing directions,
wherein the arms have a first arm and a second arm,
the arms are able to transition between a first mode in which the first arm and the second arm pivot independently, and a second mode in which the first arm, which pivots in an opening direction of the opening and closing directions, causes the second arm to pivot in a closing direction of the opening and closing directions.

20. The clip unit according to claim 19,
wherein an angle by which the first arm and the second arm open is set as an opening and closing angle,
the arms are in the first mode when the opening and closing angle is less than a predetermined angle, and
the arms are in the second mode when the opening and closing angle is equal to the predetermined angle.

21. The clip unit according to claim 16,
wherein the first arm has a link portion that engages with the second arm when the arms are in the second mode.

22. The clip unit according to claim 17,
wherein the first arm is supported by the holder to be pivotable about a pivot axis, and
a distal end of the first arm and the link portion of the first arm are provided on opposite sides of the pivot axis.

23. The clip unit according to claim 21,
wherein the second arm, which pivots in the opening direction of the opening and closing directions, is caused to pivot in the closing direction of the opening and closing directions by the first arm in the second mode, and
the second arm has a link portion that engages with the first arm when the arms are in the second mode.

24. The clip unit according to claim 17,
wherein the second arm is supported by the holder to be pivotable about a pivot axis, and
a distal end of the second arm and the link portion of the second arm are provided on opposite sides of the pivot axis.

25. The clip device according to claim 19, further comprising:
a connecting member configured to detachably connect the sheath and the holder,
wherein the connecting member separates the sheath and the holder when a wire connected to the arm is pulled.

26. The clip device according to claim 19, further comprising:
a locking mechanism that locks the arms in a closed mode.

27. The clip device according to claim 26,
wherein the locking mechanism includes an engaged portion provided on the holder and an engaging portion provided on the arms, and
when the arms are pulled by the wire connected to the arms, the engaging portion engages with the engaged portion, thereby locking the arms in the closed mode.
